(19) United States
(12) Patent Application Publication
(10) Pub. No.: US 2019/0194646 A1
(43) Pub. Date: Jun. 27, 2019

(54) RAPID AND SIMPLIFIED DEVELOPING ANTIBODIES AND FABs OR OTHER MOLECULES CONSISTING OF AMINO ACIDS RECOGNIZING NATURAL ANTIGENS OR NON-NATURAL MOLECULAR TARGETS USING SPECIAL BLOCKING AGENT

(71) Applicant: Aleksandr Faynboym, Daly City, CA (US)

(72) Inventor: Aleksandr Faynboym, Daly City, CA (US)

(21) Appl. No.: 15/849,664

(22) Filed: Dec. 21, 2017

(51) Int. Cl. C12N 15/10; C40B 40/02; C07K 16/00; C07K 1/04; C07K 14/47

(52) U.S. Cl. CPC C12N 15/1037; C40B 40/02; C07K 16/005; C07K 2317/55; C07K 14/47; C07K 2319/035; C07K 1/047

(57) ABSTRACT

A rapid and simplified method of developing antibodies or other monoclonal antigen-recognizing polypeptides comprised of amino acids (polypeptides capable of forming complexes with targeted antigens, comprised of natural or non-natural molecular targets), using a Bioparticle Display Library approach, with each bioparticle of the Bioparticle Display Library containing gene coding polypeptide and capable of multiplying in the presence of some particular multiplication limiting factor. A polypeptide-carrying bioparticle insulated from a complex mixture of similar bioparticles of the Bioparticle Display Library using a first Blocking Agent, said Blocking Agent consisting of a bioparticle strain which lacks resistant to this multiplication limiting factor (for example for Phage Display Library multiplication limiting factor can be some antibiotic, and blocking agent would be phage strain which is lacks resistance to this particular antibiotic).

### CROSS-REFERENCE TO RELATED APPLICATION(S)

This application claims priority from U.S. Provisional Application 62/380,461 filed Aug. 28, 2016, which is incorporated herein.

### BACKGROUND OF THE INVENTION

### 1. Field of Invention

The present invention relates to the field of antibodies, and more particularly, the embodiments of the present invention relate to a rapid and simple developing of antibodies and fabs (mono- or polyclonal) or other molecules consisting of amino acids recognizing natural antigens or non-natural molecular targets.

### 2. Description of Related Art

Modern medicine increasingly relies and requires pharmaceutical and biotechnological manufacturing techniques for creation of therapeutic and diagnostical antibodies.

Since the 1970's when monoclonal antibodies and DNA cloning were discovered, the biotechnology industry has amassed $900 billion. See Forbes May 5, 2015. Total pharma revenues are expected to increase from $1.23 trillion in 2014 to $1.61 trillion in 2018. See http://www2.deloitte.com/content/dam/Deloitte/global/Documents/Life-Sciences-Health-Care/gx-lshc-2015-life-sciences-report.pdf). 2012 sales value of just seven monoclonal antibodies reached US $20 billion. The quality of new drugs (NME/BLA) approved by the FDA in 2013 increased by 43%, versus 2012, with nine of the top ten forecast to reach blockbuster status—$1 bn sales in the US—five-years post launch. And, 2013 became the best year ever for new drug approvals. This exceeds the record breaking year of 2012 and marks a step-change in innovation and output for the industry since 2009. In total, the class of 2013 was expected to add over $24.4 bn to total US prescription drug sales in 2018.

There are 2,500 biotech Companies in USA [in 2013, private biotech companies raised 5.7 billion U.S. dollars of capital, while public companies raised some 20 billion U.S. dollars “Statista”-statistic portal (Financing data of U.S. biotech companies from 2012 to 2014)], 250 Universities and 200 medical-biological Institutions each of which contains from few to few hundred R&D laboratories or departments where practically every researcher needs specific antibodies for their particular scientific purposes.

“EvaluatePharma® World Preview 2014, Outlook to 2020” forecasts from 2014 to 2020 that: “we have an extended view of how equity analysts are modelling the impact of biological patent expiries and the subsequent entry of bio-similar products: they continue to expect a softer landing, post patent expiry and limited bio-similar substitution. It seems future metaphorical patent cliffs are being transformed into much more manageable rolling hills. R&D expenditure is forecast to grow at a modest 2.4% per year between 2013 and 2020, reaching $162 bn.

The growth of R&D is below sales growth and continues the strategic industry theme of cost containment. In the last 3 to 4 years, every Pharmaceutical symposium has discussed problem of precise tuning-up specificity of Monoclonal Antibodies, which should be achieved for special needs in clinical use of Monoclonal Antibodies candidates for therapeutic Monoclonal Antibodies. “Monoclonal Antibody Therapeutics: The Need for Biopharmaceutical Reference Materials” John E. Schiel*, Anthony Mire-Sluis, Darryl Davis National Institute of Standards and Technology, Gaithersburg, Md. 20899, US; North America, Singapore, Abingdon, Amgen Inc., Thousand Oaks, Calif. 91320, US Janssen Research and Development, LLC, Spring House, Pa., United States ACS Symposium Series, Vol. 1176 ISBN13: 9780841230262eISBN: 9780841230279 Publication Date (Web): Dec. 16, 2014 Copyright © 2014 American Chemical Society, etc. Commercially available antibodies or an antibody in which the company's own the relevant R&D provides a very low percentage of good quality and suitable antibodies for clinical use. Emerging personalized medicine and immunotherapeutic approaches need much more precise characterization of metabolic status and therapeutic antibodies. This task is only possible to achieve with new immune tools which does not exist yet. This is a problem which health science and pharmacology are trying to address with present and near future international and local symposiums: (Annual Meeting EB 2017/Apr. 24, 2017; CHI Apr. 18, 2017; 6th Antibody & Protein Therapeutics Summit Boston, Mass. Jun. 28, 2018 and many more). The disclosed embodiments are intended to address the above concerns and problems and contain improvement of present day techniques with a progressive approach to get a diversified pool of Monoclonal Antibodies using gene engineering based libraries for getting antibodies.

Prior art, classical methods developing antibodies were based on using mammalian animals or avian immune systems which consists of 10⁸ (100,000,000) variants of immune molecules called antibodies. It is time and very labor intensive as well as not always reproducible method. Gene engineering based modern approach—Polypeptides Display Libraries—has already proved to be more effective. Present day methods are using gene-engineered artificial “immune systems” which are showing faster process and wider variety of resulted polypeptides.

It consists of few approaches where genetic information bounded with coded polypeptides displayed on a surface of different Bio-particles. “Biological Particles Display Library” (BP-DL) in which used artificial “immune systems” created by gene-engineering methods and Polypeptides are Displayed on surfaces of Different Biological (containing their genes) Particles: different cells (yeast, bacterial or mammalian), phages or ribosomes. In correlation with their nature its names are: Phage Display (Ph-DL), yeast display (Y-DL), Bacterial display (B-DL), Ribosome display (R-DL), mRNA display (RNA-DL) and mammalian cell Display (M-DL) Libraries. Different variants will be appearing but all of them will have common feature: they will contain genes (DNA or RNA based) of some polypeptides with randomly variable sequence coding polypeptides displayed on surface of biological particles. Here, these technics can be referred to as “Bioparticles Display Libraries (BP-DL)” because the idea is applicable for all of them. Despite it when it is needed to use in some examples to describe some details “Phage Display Library” is used as example because it appeared the very first and has the widest use in modern art of achieving for researchers important Antibodies or other antigen-recognizing Polypeptides.

The modern-day approach—Bioparticles Display Library (for example “Phage Display Library”)—consists of 10¹^{⁰}-10¹² (10,000,000,000-1,000,000,000,000) individual variable polypeptides (ScFv or others) or small double chain Fragments of Antibodies (FABs) which are displayed on the surface of Bioparticles (for example phages). Other BpDL has similar or less complexity. Choosing useful (specifically binding target molecule) polypeptide-carrying bioparticles (phages) out of “Bioparticles (Phage) Display Library” is based on screening polypeptides displayed on the surface of Bioparticles (phages) with an ELISA-Like process known as “Panning.”

This process (PANNING) consists of PREPARING Antigen for immobilization or separation; SATURATING (blocking) not occupied immobilization sites; INCUBATION of polypeptide-carrying bioparticles (phages, yeasts or ribosomes) at presence of Blocking solution; WASHING OUT UNBOUNDED bioparticles; ELUTION of BOUNDED bioparticles. Later MULTIPLICATION of eluted bioparticles (infection of bacterial or other strains carrying some Antibiotic Resistance or other MULTIPLICATION limiting agent) follows resulting in separating some clones carrying polypeptides specifically recognizing target molecules. Then SCREENING of clones containing bioparticles bearing SPECIFIC polypeptides on its surface which can recognize chosen by researcher target (see size comparison FIG. 1). Clones then should be recloned.

In parallel with “SPECIFIC” bioparticles this process is catching a lot of “NONSPECIFIC” (which does not carry polypeptide specifically recognizing chosen target) bioparticles. That is why the regular method includes “panning” of Phage Display Library in presence (“positive probe”) and absence (“negative probe”) of target of interest. Experiments could be arranged using biotinylated antigens and later Biotin-capturing technic or with immobilized on some surface antigens.

Every cycle of PANNING and MULTIPLICATION with and without antigen-containing probe is called a “ROUND”. Every SCREENING regularly consists of 3-5 “ROUNDS”. After every “ROUND” number of “colony forming units” or “positively labeled ribosomes” eluted from “positive” and “negative” probes should be compared. When amount clones from “positive” probe become bigger than amount clones from “negative” probe DETECTING of specifically binding target molecule STARTS as well as specific SELECTION.

During every “ROUND of selection” not only researcher-directed (expected) selection appears, but also appears selection based on the nature of phage (undirected by researcher): effectiveness of its bacteria-transforming abilities (or yeast cells growth rate), its relationship with bacteria and nature of that bacteria cell transformed with phage. Also, some bioparticles (for example phages retained just by its difference in shape from blocking molecules. Some colonies of phage infected bacteria are growing faster, some are growing slower, without correlation with key features of those peptides which phages are bearing and are being searched for.

Each bacterial (or cell) colony contains number of cells differs 10-100 fold which means (because every cell develops one colony during each round) after three rounds some fast growing clones and their surface-displayed specific peptides become 1000-1,000,000 times more represented than slow growing (but very possible more useful by key feature of polypeptides which they are bearing) competitors. And, all these negative processes are happening before first directed SELECTION become possible.

Based on the foregoing, there is a need in the art a system, which will facilitate getting a diversified pool of suitable Monoclonal Antibodies—improvement of “Phage Display Library” method—for a desired purpose.

### SUMMARY OF THE INVENTION

A rapid and simplified method of developing antibodies or other monoclonal antigen-recognizing polypeptides comprised of amino acids (or artificial amino acids) and capable of recognizing and forming complexes with targeted antigens, comprised of natural or artificial molecular targets, using a Bioparticle Display Library approach, with each bioparticle of the Bioparticle Display Library containing genes composed of nuclear acids and capable of multiplying in the presence of a multiplication limiting factor. A polypeptide-carrying bioparticle identified and separated from a complex mixture of similar bioparticles of the Bioparticle Display Library using a first blocking agent, said first blocking agent consisting of a bioparticle, carrying only one individual type of polypeptide or not carrying any type of polypeptide, and resistant to different ones of the Bioparticle Display Library antibiotic or some other multiplication-limiting factor, or lacks resistance to any antibiotic, or to other multiplication-limiting agents. Performing an only single round of panning, multiplication, and screening using the blocking polypeptide-carrying bioparticle and immunoassays to generate visible indicator of a desired biologic property.

Producing and selling kits of reactives for producing the antibodies or the polypeptides capable of recognizing natural antigens or non-natural molecular targets containing the first blocking agent.

Producing, using, and selling separately a plurality of blocking agents comprised of the first blocking agent and blocking bioparticles to block nonspecific binding of bioparticles of a Bioparticle Display Libraries for producing antibodies, small double chain Fragments of Antibodies (FABs), and other polypeptides capable of recognizing natural antigens or non-natural molecular targets.

The used Bioparticle Display Library comprises at least one of a phage-, mammalian or yeasts cell-, ribosome-, or RNA-Display Library.

The multiplication limiting factor comprises a particular antibiotic, some other multiplication-limiting factor, or a factor disturbing the bioparticle some way to be involved in the round.

The visible indicator comprises a color or size of a bacteria colony on a Petri dish or bacteria present and not forming colonies.

After the single round, performing an enzyme-linked immunosorbent assay (ELISA) verification, then performing a binning using epitope comparison, and then performing specialized analyses.

The specialized analyses comprise immunohistochemistry (IHC), Ligand-Receptor Binding, Enzymatic Activities, Immune assays of specific pathological collection of samples etc.

Thus, an object of the embodiments of the present invention is to provide a rapid and simple developing of single chain polypeptides or FABs or other molecules consisting of amino acids recognizing natural antigens or non-natural molecular targets, which avoids the disadvantages of the prior art.

Thus, an object of the embodiments of the present invention is to provide a rapid and simplified developing MABs or single chain polypeptides or other molecules consisting of amino acids recognizing natural antigens or non-natural molecular targets, which avoids the disadvantages of the prior art.

Briefly stated, object of the embodiments of the present invention is to provide a rapid and simple developing single- or double-chain polypeptides (mono- or oligoclonal) or other molecules consisting of amino acids capable of recognizing (forming complex) natural antigens or non-natural molecular targets which could be singled out and separated from mixture of 1:1,000,000,000,000 or more using special Blocking Reagent. The Blocking Reagent blocks Nonspecific binding of phages (or other bioparticles) from Phage- (or Yeast-, or Ribosome-like, or cells-) Display Libraries resistant to some antibiotic (or any other MULTIPLICATION-limiting agent) to immobilized or labeled molecular targets with some Phage (or Yeast-, or Ribosome-like, or cells-) strain which have NO or some OTHER antibiotic resistance (or not in compliance with other MULTIPLICATION-limiting agent).

The foregoing, and other features and advantages of the invention, will be apparent from the following, more particular description of the preferred embodiments of the invention, the accompanying drawings, and the claims. The novel features considered characteristic of the embodiments of the present invention are set forth in the appended claim. The embodiments of the present invention themselves, however, both as to their construction and to their method of operation together with additional objects and advantages thereof will be best understood from the following description of the embodiments of the present invention.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 demonstrates imaging of Phage-Antigen specific & nonspecific interaction; comparison sizes and shapes of different blocking agents (using phages as example) according to an embodiment of the present invention;

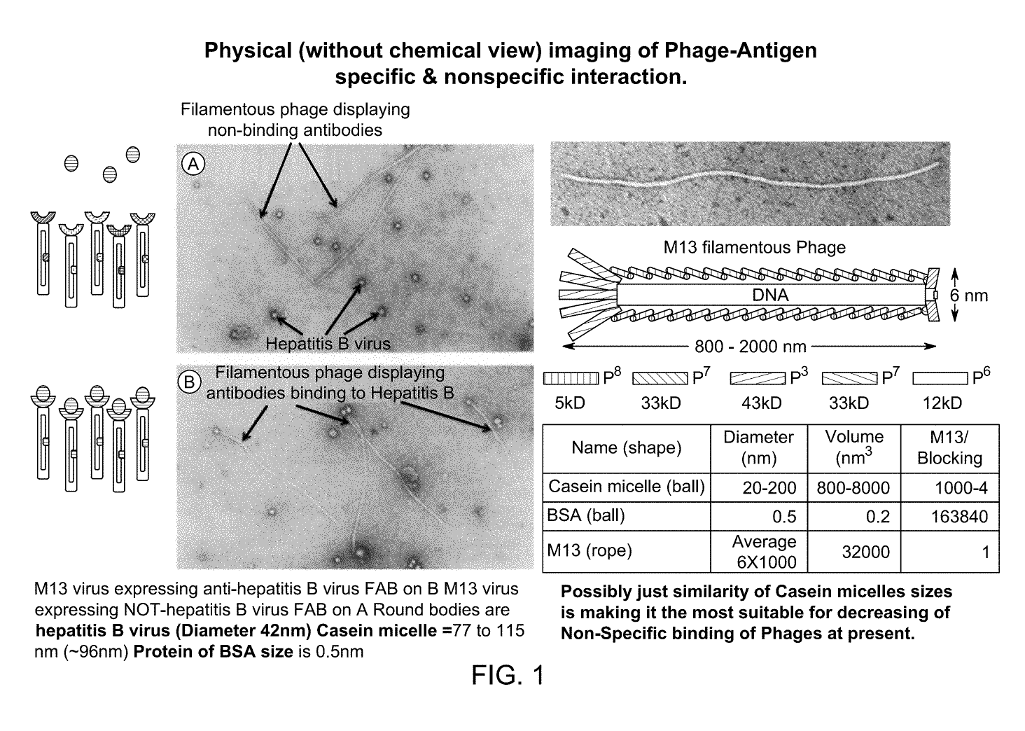

FIG. 2 demonstrates a comparison of new versus classical approach of clone screening, according to an embodiment of the present invention.

### DETAILED DESCRIPTION OF PREFERRED EMBODIMENTS

Embodiments of the invention are discussed below. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

Headings provided herein are for convenience and are not to be taken as limiting the disclosure in any way.

The enumerated listing of items does not imply that any or all of the items are mutually exclusive, unless expressly specified otherwise.

The terms “a”, “an” and “the” mean “one or more”, unless expressly specified otherwise.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

The present invention will now be described in detail with reference to embodiments thereof as illustrated in the accompanying drawing.

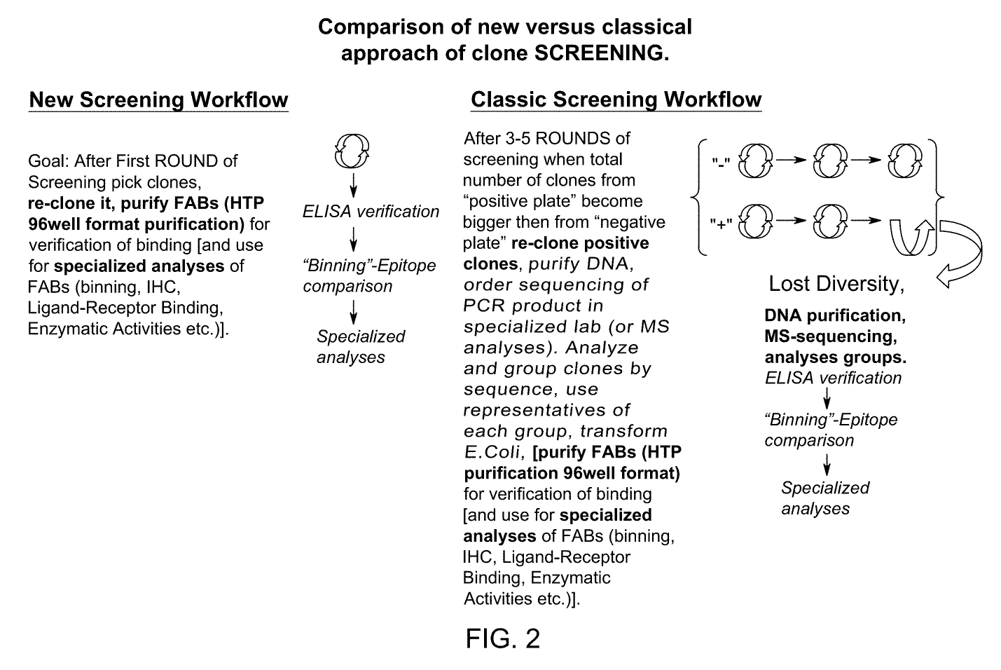

FIG. 2 provides a comparison of old and new screening processes.

The new process details shown for (Phage Display Library): phage particles (or other Bioparticles) not carrying particular antibiotic resistance (or not in compliance with other limiting MULTIPLICATION agent) can block non-specific binding of the phages (or other Bioparticles) of Phage Display Library (or other Bioparticles-Display Library) with particular antibiotic (or which is in compliance with other limiting MULTIPLICATION agent) resistance because of its physical-chemical similarity but incapable to be involved in MULTIPLICATION step of ROUND and that is why cannot make disturbance in SCREENING.

Blocking should be performed before and (or) during panning with strain of Blocking phage (Bioparticle) which does not have resistance to any antibiotic [or for convenience to work with ones which have resistance to another antibiotic (or which is in compliance with other limiting MULTIPLICATION agent)]. Which means these phages (Bioparticles) cannot participate in the MULTIPLICATION step (bacteria clones infected with these phages cannot create colonies on Petri dishes with chosen antibiotics because they lack resistance against this antibiotic). For example, if the Phage Display Library has Ampicillin and/or Kanamycin resistance; the selected blocking phage should have Tetracycline or Chloramphenicol resistance (or none).

Blocking non-specific binding of phages (Bioparticles) of Phage Display Library (Bioparticles-Display Library) which carry resistant against some antibiotic (or in compliance with some MULTIPLICATION limiting factor) to immobilized or tagged molecular targets could be performed with Blocking phage (Blocking Bioparticle) strain which have no or some other antibiotic resistance (or not in compliance with used MULTIPLICATION limiting factor) is a novel approach. During the MULTIPLICATION step on the Petri dish, in the presence of a chosen antibiotic (or other MULTIPLICATION limiting factor), bacteria cells will develop colonies only if they are transformed with phage which carry resistance for the chosen antibiotic, but not non-specifically bounded phages which will be substituted with the Blocking phage, which does not have this resistance, and cells transformed with these phages will not develop bacteria colonies on the Petri dish with the chosen antibiotic present.

A new screening workflow: performs a first round of Phage (or another Bioparticle) Display Library SELECTIION.

PREPARATION OF TARGET MOLECULE: immobilization target molecule on surface of 96 well plastic plate or labeling target molecule with some tag (for example Biotin) which possible to use for following separation. After immobilization of target molecule (1-100 μg/ml for regular size protein), it is necessary to remove (wash out) free (not immobilized or untagged) molecules. Because immobilization or labeling cannot be in saturation mode, you need to block sites still capable to nonspecifically bind any molecules with blocking solution (regular it is 2% Dry Milk in 20 mM Phosphate Buffer pH 7.4, 150 mM NaCl (PBS) with 0.1%-0.2% Tween-20, in presence or absence of 1-10 mg/ml Bovine Serum Albumin and—Blocking phage 10¹¹ phage particles per well).

INCUBATING—adding the desired Phage Display Library with some antibiotic resistance (10¹⁰-10¹¹ per well) to immobilized antigen [after saturating (blocking not occupied immobilization sites) with blocking solution (regular it is 20 mM Phosphate Buffer, 150 mM NaCl (PBS) with 0.1% Tween-20, in presence or absence of 1-10 mg/ml Bovine Serum Albumin and—Blocking phage approximately 10¹¹-10¹² phage particles per well). After allowing the phage particles time to bind (in presence of the Blocking phage in a blocking solution) follows—

WASHING OUT—regularly conducted with PBS-Tween solution 10-20 times. Phages which are displaying polypeptide that binds to the target molecules become immobilized on the surface of well of 96 well plate (or to tagged target molecule for future separation) remain attached to the surface of the well, while others are washed away.

ELUTION—treatment of the Phages (regular with denaturating agents like acidic buffer pH2.2 with following neutralization or by competitive substitution by free target molecule or its fragments) to elute the bounded phages out to use in following multiplication.

MULTIPLICATION (the desired eluted phages are used for infection bacteria cells, then bacteria cells are growing in presence of that antibiotic against which phages of Phage Display Library has resistance. Infected with phages from Phage Display Library bacteria cells are capable to form colonies on Petri dishes in presence of for example antibiotic Ampicillin while uninfected cells or infected with Blocking phage which does not carry resistance against Ampicillin can NOT grow and form colonies. In regular method after the first ROUND, the selected phages have been multiplied in infected bacteria together with non-specifically caught phages from the same Phage Display Library and Round was repeated few times losing on the way 99%-99.9% of slow growing colonies carrying a majority of useful phages. With singe Round screening instead of repeating Rounds a researcher picks clones after first ROUND using ECL, Fluorescent or Radio detection of Petri dishes nitrocellulose replica, ELISA and other High Throughput assays, based on chosen features from undepleted variety of target recognizing polypeptides and then reclone chosen clones the same way like in classical workflow, next follow ELISA or other methods VERIFICATION, followed by BINNING for epitope comparison, and then different SPECIALIZED methods of ANALYSES.

In an embodiment, VERIFICTION can be accomplished by ELISA. The enzyme-linked immunosorbent assay (ELISA) uses antibodies and color change to identify a substance, and it a popular format of “wet-lab” type analytic biochemistry assay that uses a solid-phase enzyme immunoassay (EIA) to detect the presence of a desired antigen or protein in a liquid sample or wet sample. ELISA has been used as a diagnostic tool in medicine and plant pathology, as well as a quality-control check in various industries more than 55 years. Now very often its usage is limited and for many tasks it is necessary to use very specialized methods (FACS, Immunohistostaining (HIS), ligand assays etc.).

Antigens or proteins from the sample are attached to a surface. Then, a further specific antibody is applied over the surface so it can bind to the antigen or protein. This antibody is linked to an enzyme, and, in the final step, a substance containing the enzyme's substrate is added. The subsequent reaction produces a detectable signal, most commonly a color change in the substrate.

Typically, performing an ELISA involves at least one antibody with specificity for a particular antigen. The sample with an unknown amount of antigen is immobilized on a solid support (usually a polystyrene microtiter plate or nitrocellulose film) either non-specifically (via adsorption to the surface) or specifically (via capture by another antibody specific to the same antigen, in a “sandwich” ELISA). After the antigen is immobilized, the detection antibody is added, forming a complex with the antigen. The detection antibody can be covalently linked to an enzyme, or can itself be detected by a secondary antibody that is linked to an enzyme through bioconjugation. Between each step, the plate is typically washed with a mild detergent solution to remove any proteins or antibodies that are non-specifically bound. After the final wash step, the plate is developed by adding an enzymatic substrate to produce a visible signal, which indicates the quantity of antigen in the sample such as color or size of the colonies.

Clones will be picked according to the processes of detection described above or many others specific for every research lab or group, and then physically separated on Petri dishes. Chosen Colonies will then be picked up and seeded on other smaller plates with the same antibiotic (called RECLONING), to re-clone the clones (separate from neighbor clones), and then purify polypeptides for verification of binding characteristics, and use for specialized analyses of FABs (binning, FACS, immunohistochemistry (IHC), Ligand-Receptor Binding, Enzymatic Activities, Immune staining of specific pathological collection of samples etc.) to pick out the desired polypeptide-carrying phages from separated bacteria colonies to clone, propagate and purify. Interesting FABs later could be converted to MABs (monoclonal antibodies), bivalent antibodies; immunotoxins etc.

In this application, the PANNING in presence one of Blocking phage (Blocking Bioparticle) strain which do not carry antibiotic resistance chosen for Phage Display Library done in the single round gives rise to an easily, visually detectable set of bacteria colonies containing phages exhibiting the desired characteristics, which are picked, cloned and re-cloned. Typically, these sets of colonies are contaminated with millions of non-specifically bounded phages (which give rise of millions of bacteria colonies) carrying the same antibiotic resistance but not-carrying peptides specifically exhibiting the desired characteristics and binding target molecule nonspecifically. The new approach substitutes these contaminating phages with Blocking phages, which do not carry the same antibiotic resistance and do not give rise to bacteria colonies in a solution or on Petri dish in presence of this antibiotic (no MULTIPLICATION).

In an embodiment, after ELISA verification, “Binning” for EPITOPE COMPARISON to accomplish grouping of polypeptides is performed. Epitope binning is a competitive immunoassay that characterizes and then sorts the library of monoclonal antibodies against the target antigens or protein. Antibodies against a similar target can also be tested against other antibodies in the library in a pairwise fashion to see if antibodies block one another's binding to the epitope of an antigen. For the analysis, each antibody has a profile created against all of the other antibodies in the library, and a competitive blocking profile is created for each antibody relative to the others in the library. Closely related binning profiles indicate that the antibodies have the same or a closely related epitope and are “binned” together.

After the epitope comparison, a SPECIALIZED ANALYSES can be performed. During the specialized analysis of FABs using binning, Immune Histochemistry (IHC), FACS (Fluorescence-activated cell sorting), Target-Receptor Binding, known or unknown Enzymatic Activities. At the end, a researcher has FABs (polypeptides) with the desired characteristics to use which can be converted to MABs or other immune molecules.

In an embodiment, the above technique can be implemented by pre-designed kits made up of reagents containing some Phage Display Library and particular Blocking Agents containing Phage with different antibiotic (or other limiting MULTIPLICATION agent) resistance (or without any resistance) than the antibiotic Resistance of Phage Display Library, allowing customers to get antibodies of their choice during 3-4 days without labor-consuming work or ordering any analyses by specialized companies.

In an embodiment, the invention provides a rapid and simplified method of developing antibodies or FABs (mono- or oligoclonal) or other molecules consisting of amino acids recognizing natural antigens or non-natural molecular targets. The polypeptides capable of recognizing molecular targets could be singled out and separated from a mixture of 10¹¹-10¹² [10,000,000,000-1,000,000,000,000] (or more) other unique polypeptides from polypeptide-carrying Biological Particles (phages-, yeasts-, ribosomes-, cells-) Display Libraries capable to MULTIPLY in presence of some MULTIPLICATION-limiting factor using a special Blocking Agent.

The composition (Blocking Agent: biological Particle incapable of being involved one way or another in MULTIPLICATION step of SELECTION) blocks nonspecific binding of the biological Particles Display Library (which is resistant to some antibiotic or other MULTIPLICATION limiting factor) to immobilized or labeled molecular targets with some particle strain which have no or has some other antibiotic resistance to another antibiotic or to other limiting MULTIPLICATION agent).

In an embodiment, Blocking Agent for producing antibodies, FABs and other polypeptides capable of recognizing natural antigens or non-natural molecular targets can be offered in kits.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

I claim:

1. A rapid and simplified method of developing antibodies or other monoclonal antigen-recognizing polypeptides, comprising:
amino acids comprising polypeptides capable of recognizing and forming complexes with targeted antigens, comprised of natural or artificial molecular targets, using a Bioparticle Display Library approach, with each bioparticle of the Bioparticle Display Library containing genes composed of nucleic acids and capable of multiplying in the presence of a multiplication limiting factor;
a polypeptide-carrying bioparticle identified and separated from a complex mixture of similar bioparticles of the Bioparticle Display Library using a first blocking agent, said first blocking agent consisting of a bioparticle, carrying only one individual type of polypeptide or not carrying any type of polypeptide, and resistant to different ones of the Bioparticle Display Library antibiotic or some other multiplication-limiting factor, or lacks resistance to any antibiotic, or to other multiplication-limiting agents; and
performing only a single round of panning, multiplication, and screening using the blocking polypeptide-carrying bioparticle to generate a visible indicator of a desired biologic property.

2. The rapid and simplified method of developing antibodies or other monoclonal antigen-recognizing polypeptides of claim 1, further comprising:
producing and selling kits of reactives for producing the antibodies or the polypeptides capable of recognizing natural antigens or non-natural molecular targets containing the first blocking agent.

3. The rapid and simplified method of developing antibodies or other monoclonal antigen-recognizing polypeptides of claim 1, further comprising:
producing, using and selling separately a plurality of blocking agents comprised of the first blocking agent and blocking bioparticles to block nonspecific binding of bioparticles of a Bioparticle Display Libraries for producing antibodies, small double chain Fragments of Antibodies (FABs), and other polypeptides capable of recognizing natural antigens or non-natural molecular targets.

4. The rapid and simplified method of developing antibodies or other monoclonal antigen-recognizing polypeptides of claim 1, wherein the used Bioparticle Display Library comprises at least one of a phage-, mammalian or yeasts cell-, ribosome-, or RNA-Display Library.

5. The rapid and simplified method of developing antibodies or other monoclonal antigen-recognizing polypeptides of claim 1, wherein the multiplication limiting factor comprises a particular antibiotic, some other multiplication-limiting factor, or a factor disturbing the bioparticle involved in the round.

6. The rapid and simplified method of developing antibodies or other monoclonal antigen-recognizing polypeptides of claim 1, wherein the visible indicator comprises a color or size of a bacteria colony on a Petri dish or bacteria present and not forming colonies.

7. The rapid and simplified method of developing antibodies or other monoclonal antigen-recognizing polypeptides of claim 1, further comprising:
after the single round, performing an enzyme-linked immunosorbent assay (ELISA) or membrane-based assay verification;
then performing a binning using epitope comparison; and
then performing specialized analyses.

8. The rapid and simplified method of developing antibodies or other monoclonal antigen-recognizing polypeptides of claim 1, wherein the specialized analyses comprises of immunohistochemistry (IHC), Ligand-Receptor Binding, Enzymatic Activities, Immune assays of specific pathological collection of samples or other immunological, biochemical or biological assays.
